(19) United States
(12) Patent Application Publication
(10) Pub. No.: US 2025/0281409 A1
(43) Pub. Date: Sep. 11, 2025

(54) CANNABINOIDAL COMPOSITIONS AND METHODS OF PRODUCING THE SAME

(71) Applicant: 113 Botanicals Limited, Southend On Sea (GB)

(72) Inventors: Mazin NICOLA, Worthing (GB); Daniel GUEST, Little Worth Partridge Green (GB)

(21) Appl. No.: 18/861,405

(22) PCT Filed: Apr. 12, 2023
(86) PCT No.: PCT/GB2023/050984

§ 371 Date: Oct. 29, 2024

(30) Foreign Application Priority Data
Apr. 29, 2022 (GB) 2206268.1

(51) Int. Cl. A61K 9/16; A61K 9/48; A61K 31/00; A61K 36/185

(52) U.S. Cl. CPC A61K 9/1635; A61K 9/1652; A61K 9/4891; A61K 31/658; A61K 36/3482; A61K 2236/35

(57) ABSTRACT

Compositions and Methods A method of producing a medicinal composition comprising an extract of cannabis plant material comprises: (i) mixing, for example dissolving, an extract of cannabis plant material in a liquid vehicle to form a liquid mixture: (ii) contacting the liquid mixture with a first excipient: (iii) contacting the combination comprising the liquid mixture and said first excipient with a form modifying material, for example a coating material, to form a precursor composition; and (iv) treating, for example pelletising, the precursor composition to form the medicinal composition.

This invention relates to compositions and methods. Preferred embodiments relate to compositions, for example in predetermined unit dosage forms, of extracts from cannabis plant material, such as from Cannabis sativa. Preferred embodiments relate to medicinal compositions comprising cannabinoids and/or terpenes and methods of making such compositions.

Cannabis Sativa is believed to be one of the oldest plants cultivated by man. The plant and preparations therefrom have been used for its medicinal properties for many centuries, although uses are prohibited by laws in many countries. The easing of some of the prohibition laws relating to the cultivation and use of cannabis material started with “California Proposition 215” also known as The Compassionate Use Act in 1996 and by 2016 medical use of cannabis was legalised in most of the USA. This resulted in the ever-increasing global demand for cannabis plant products for recreational use and as health supplement and homeopathic medicine.

Worldwide governmental restrictions on cultivation, use of and research into cannabis meant that clinical process research work has not been sufficiently rigorous. Therefore, there exists a real need to work towards clearer understanding and development of the therapeutic activities of cannabis-based products and the development of optimum routes of administration for the purposes of efficacy and safety.

Although much of the research carried out to date has been directed towards the bioactivity of the phytocannabinoids, emphasis is increasingly being placed on the full spectrum of phytochemicals including terpenes and phenylpropanoids.

Cannabis based medicinal products (CBMPs) are defined as mixtures comprising one or more purified cannabinoids including THC, CBD and others. They are manufactured to Good Manufacturing Practice (GMP) standards and are compliant with all patient safety requirements. Only two CBMPs, based on purified isolates, currently have full market authorisation. In order to have market authorisation an unlicensed CBPM has to go through a New Drug Application (NDA) process. This is a long and tortuous process that starts with a formal application to the regulatory body and include full pharmacodynamic data and pharmacokinetic data as well as description of the manufacturing processes, analytical data relating to product purity and specification, and impurity profiles, batch-to-batch reproducibility and efficacy.

While cannabinoids and terpenes have been studied substantially for their individual therapeutic effects, there is a general consensus that CBMPs activate their pharmacological effects in the living body via synergistic/antagonistic interactions between the various phytochemicals that are present in the cannabis-based products and the endocannabinoid system (ECS). This phenomenon, also known as “entourage effect”, was coined to explain why botanical drugs containing the entire spectrum of compounds within a plant can be more effective than the plant's isolated components. A good example of the entourage effect is the stronger muscle-antispastic effect of cannabis extract compared to pure THC, this represents an important finding for the treatment of multiple sclerosis.

It is generally accepted by medical practitioners that cannabis biomass and/or extracts from cannabis biomass have important therapeutic benefits. However, general practitioners (GPs) in the UK can be reluctant, or feel unable, to prescribe CBMPs for a range of reasons. A recent survey of over 1000 GPs, carried out by the Primary Care Cannabis Network (PCCN), titled “UK GP attitudes towards medicinal cannabis survey 2021”) a summary of which shows that 73% of respondents are open minded about CBMPs furthermore, over 50% supported the use of CBMPs as alternatives for patients who have exhausted licensed medicines whilst and less than 15% of respondents believe that CBMPs were more hazardous than opioids, Benzodiazepines, Gapapentinoids and Z-drugs (Zopiclone, zaleplon, eszopiclone and zolpidem) in the treatment of a number of conditions including of chronic pain, anxiety, restless leg syndrome, spasms and insomnia. Nevertheless, only 24% of respondents said they would prescribe CBMPs as alternatives for the treatment of the above listed conditions and 76% said they are not likely to do so under current circumstances.

Figures published by Public Health England (September 2019: Authors: Stephen Taylor et al) show that the total combined number of prescriptions of opioids. Benzodiazepines, Gapapentinoids and Z-drugs (Zopiclone, zaleplon, eszopiclone and zolpidem) for that year alone were over 130,000,000 and the number of patients who received a prescription, continuously, for a minimum period of 5 years exceeded 1,850,000. Patients using these drugs routinely experience severe and often none-reversible physical, emotional, social and sexual side effects (Addictive Behaviours, Volume 125, February 2022, J. Davies et al) Opioid use contributes to over two-thirds of all drug overdose-related deaths in the USA in 2017-2018 (MMWR Morb Mortal Wkly Rep 2020; 69:290-7. Wilson N, et al).

One problem which is very relevant to whether GPs prescribe cannabis is in selecting an appropriate dosage form or a safe way of delivering the cannabis active ingredients. Free-text comments by respondents in the PCCN survey (referenced above), explaining the reasons for reluctance of clinicians to prescribe CBMPs as alternatives to these often over-prescribed and problematic medications, in addition to lack of knowledge and education, included concerns over potential abuse, addiction and dependency, costs of treatment, potential psychological side effects, interaction with other medications and general imprecise formulary and high risk of accidental overdosing.

Whilst use of THC and/or CBD-based medicinal dosage forms are available, they tend not to be full spectrum cannabis extracts:—that is, they may comprise one or more purified cannabinoids, rather than comprise a wide spectrum of compounds extracted from the cannabis plant. In addition, known formulations may not be slow release and/or the rate of release may not be linear with time. This may mean that a patient may receive an initial significant hit from taking a cannabis based medicinal composition, with this effect being relatively short lasting and the effect may diminish exponentially over time. As a result, a patient is more likely to take more of the composition than recommended, to get relief from an ailment and/or to replenish the initial hit.

In many cases, patients will not be able to persuade GPs to prescribe cannabis-based products at all.

In this case, patients may purchase illegal and unregulated extracts or may smoke cannabis plant material itself. In such cases, there is an increased tendency for patients to consume more than strictly necessary or appropriate to have a desired medicinal effect. This may also increase risks of dependency and addiction and progression to the use of “hard” drugs.

One embodiment of the present invention is based on the discovery by the inventors that a wide spectrum cannabis plant extract can be produced and formulated in a unit dosage form such that release of active ingredients from the dosage form is substantially linear and long-lasting. The composition may advantageously avoid initially delivering a large hit.

One object of preferred embodiments of the invention is to produce compositions comprising extracts of cannabis plants which are improved over prior art extracts.

Another object of preferred embodiments of the invention is to produce compositions which provide linear release of active ingredients which may comprise a broad or substantially full spectrum extract from a cannabis plant.

A further object of preferred embodiments of the invention is to produce compositions which provide slow release, for a period which may be up to 7 hours.

According to a first aspect of the invention, there is provided a method of producing a medicinal composition comprising an extract of cannabis plant material, the method comprising:
(i) mixing, for example dissolving, an extract of cannabis plant material in a liquid vehicle to form a liquid mixture;
(ii) contacting the liquid mixture with a first excipient;
(iii) contacting the combination comprising the liquid mixture and said first excipient with a form modifying material, for example a coating material, to form a precursor composition; and
(iv) treating, for example pelletising, the precursor composition to form the medicinal composition.

For the avoidance of doubt, an extract (or cognate expressions) includes any mass derived from or isolated from cannabis plant material. Said extract may be an active pharmaceutical ingredient (API) which may include one or a multiplicity of components.

An extract of cannabis plant material may be an extract from a member of the cannabaceae plant family. Said extract may be an extract from, for example, Cannabis sativa. Said extract may include one or more cannabinoids, for example including THC and/or CBD. Said extract may include terpenes which have been extracted from a biomass from the cannabaceae plant family, for example from Cannabis sativa.

Preferably, at least 90 wt %, more preferably at least 95 wt %, especially at least 98 wt % of said extract comprises a material which is naturally occurring in said cannabis plant material and has been extracted therefrom.

Said extract may not be a single, substantially pure material. Preferably said extract includes multiple different compounds. It preferably includes at least one of THC or CBD. It preferably includes one or more, preferably a multiplicity, of terpenes. Said extract may include at least three, preferably at least six, more preferably at least ten different compounds. For example, it may be a broad-spectrum extract from Cannabis sativa. Advantageously, it is found that, despite the fact the extract may include multiple different compounds, having multiple different solubilities, the medicinal composition is able to release components of the extract in a controlled manner which allows a patient to receive a therapeutic benefit, from a synergistic effect arising from multiple different active ingredients included in the extract. In an embodiment, the extract (eg API) may be a broad-spectrum distillate derived, for example, by distillation of a full spectrum extract. In this case, the extract may be an API which is a single purified cannabinoid (an isolate) such as CBD or THC at purities of 95%-99%.

In another embodiment, the extract (eg API) may be a trichome powder isolated from botanical biomass using mechanical means.

Said extract is preferably a liquid at atmospheric pressure and 25° C.

Said medicinal composition may be made and/or formulated to have a predetermined rate of release of said extract. For example, it may be a “slow release” or “fast release” composition. A slow release composition may be arranged to release 90 wt % of the extract over a period of more than six hours (eg downstream of a patient's stomach); a fast release composition may be arranged to release 90 wt % of the extract over a period of less than four hours (eg downstream of a patient's stomach).

Preferably, the liquid vehicle is an organic liquid (eg at STP) and, preferably, is a non-ionic organic liquid. The liquid vehicle may be in a class of emulsifiers comprising a non-ionic organic liquid. Preferably, the non-ionic organic liquid is non-toxic and is biocompatible and is acceptable for use in pharmaceutical applications. A suitable liquid vehicle for a specific extract may be determined using a solubility test.

The liquid vehicle may comprise a polyethylene glycol (PEG), a propylene glycol (PG), a polysorbate, a carboxylic acid, a mono-, di- and/or triglyceride, and/or an organic compound comprising a hydroxyl group, a carboxyl group and/or an ester group. The liquid vehicle may comprise a polysorbate. More specifically, the liquid vehicle may be a polyethylene ester with a chain of ethylene oxide units, sorbitol (or glucitol) and a primary fatty acid such as oleic acid. The liquid vehicle may have an average molecular weight between 200 g/mol and 10,000 g/mol, preferably in the range 750 g/mol to 2,500 g/mol and more preferably in the range 1,000 g/mol to 1,500 g/mol (1.0-1.5 KDalton).

The weight ratio of the extract to the liquid vehicle may vary depending upon the identity of liquid vehicle and the extract. The weight ratio defined as the weight of extract divided by weight of said liquid vehicle may be in the range 0.1 to 1, for example in the range 0.1 to 0.6.

In the medicinal composition, the sum of the weights of the extract and said liquid vehicle may represent 20-80 wt %, preferably 25-75 wt %, more preferably 30-65 wt % of the total weight of the medicinal composition.

In the medicinal composition, the weight of said liquid vehicle may represent at least 10 wt %, preferably at least 20 wt %, more preferably at least 25 wt % (and suitably less than 60 wt %) of the total weight of the medicinal composition.

In the medicinal composition, the weight of said extract may represent at least 5 wt %, preferably at least 10 wt %, more preferably at least 12 wt % (and suitably less than 25 wt %) of the total weight of the medicinal composition.

Preferably, the first excipient is an inert material. Preferably, the first excipient comprises a powder. The first excipient may comprise a cellulose, a silicate, a phosphate and/or a material with a large BET (Brunaur, Emmet and Teller) specific surface area The material may have a BET specific surface area of at least 10 m²/g, at least 50 m²/g, at least 100 m²/g or at least 200 m2/g.

When said first excipient comprises a cellulose, it may comprise a cellulose powder. Said cellulose may comprise microcrystalline cellulose (MCC), and more preferably MCC powder. The cellulose powder preferably comprises granules with a mean diameter of between 1 μm and 500 μm, more preferably between 10 μm and 250 μm or between 20 μm and 100 μm.

When said first excipient comprises a silicate and/or a phosphate it may comprise magnesium aluminometasilicate or calcium phosphate. A calcium phosphate may be a dibasic calcium phosphate. A silicate and/or a phosphate described may have a BET specific surface area as described for said first excipient.

The method, preferably step (ii) thereof, may comprises contact with said first excipient and a second excipient, wherein said first and second excipients are different. Preferably, the second excipient is an inert material. Preferably, the second excipient is a powder. The second excipient may comprise a cellulose, a silicate, a phosphate and/or a material with a large BET specific surface area. The material may have a BET specific surface area of at least 10 m²/g, at least 50 m²/g, most preferably at least 200 m²/g.

When said second excipient comprises a cellulose, it may comprise a cellulose powder. Said cellulose may comprise microcrystalline cellulose (MCC), and more preferably MCC powder. The cellulose powder preferably comprises granules with a mean diameter of between 1 μm and 500 μm, more preferably between 10 μm and 250 μm or between 20 μm and 100 μm.

When said second excipient comprises a silicate and/or a phosphate it may comprise magnesium aluminometasilicate or calcium phosphate. A calcium phosphate may be a dibasic calcium phosphate. A silicate and/or a phosphate described may have a BET specific surface area as described for said second excipient.

In a preferred embodiment, the first excipient is an AVICEL™ and said second excipient is a NEUSILIN™

The method, preferably step (ii) thereof, may comprise contact with said first excipient and/or said second excipient, and a third excipient. The third excipient may be a disintegrant. Advantageously, a disintegrant may increase the release rate of the extract. The disintegrant may comprise alginate, chitin, chitosan, or a pharmaceutically acceptable salt thereof. The third excipient may comprise a starch- or ceullulose-based excipient, or a pharmaceutically acceptable salt thereof, such as corn starch, partially pregelatinized starch, microcrystalline cellulose, and low-substituted hydroxypropyl cellulose. The third excipient is preferably a superdisintegrant. It may comprise starch glycolate, polyvinylpolypyrrolidone (PVPP), croscarmellose, chitin-silica, chitosan-silica, indion 414, mucilage of Plantago ovate, or a pharmaceutically acceptable salt thereof. The salt may comprise a sodium salt. The third excipient may comprise sodium starch glycolate, croscarmellose sodium or sodium alginate.

The method, preferably step (ii) thereof, may comprises contact with said first excipient and/or said second excipient, and a fourth excipient.

The fourth excipient may comprise a retarding agent. The fourth excipient may be a polymer arranged to sustainably release the extract. The polymer may be a polymer based on a cellulose. For instance, the polymer may be hydroxypropyl methylcellulose (HPMC) or ethyl cellulose. The polymer may be a copolymer arranged to sustainably release the extract. The copolymer may be a copolymer of ethyl acrylate, methyl methacrylate and/or trimethylammonioethyl methacrylate chloride. Preferably, the copolymer is a copolymer of ethyl acrylate, methyl methacrylate and trimethylammonioethyl methacrylate chloride. The molar ratio of ethyl acrylate to methyl methacrylate may be between 1:100 and 100:1, more preferably between 1:2.5 and 1:1.5. The molar ratio of ethyl acrylate to trimethylammonioethyl methacrylate chloride may be between 1:0.001 and 1:10, more preferably between 1:0.06 and 1:0.5. The fourth excipient may by a copolymer sold under the brand name Eudragit™.

The method may involve use of only a first and second excipient as described; or may involve use of a first, second and third excipient as described; or may involve use of a first, second and fourth excipient as described. The latter, in particular, may be used to produce relatively fast release medicinal compositions.

The sum of the wt % of excipients, for example, said first, second, third and/or fourth excipients in said medicinal composition may be at least 40 wt %, preferably at least 50 wt %. The sum may be less than 85 wt % or less than 80 wt %.

Preferably, the form modifying material, for example said coating material, is an inert material. Preferably, the form modifying material comprises a powder. The form modifying material may have a BET specific surface area of at least 10 m²/g, at least 50 m²/g, or at least 200 m²/g. The form modifying material may comprise silica, magnesium aluminometasilicate or calcium phosphate. The silica may comprise fumed silica.

The form modifying material, for example said coating material, is preferably a silica, for example a fumed silica. Preferably, the amount of form modifying material used is sufficient to cause the medicinal composition to comprise at least 0.25 wt % form modifying material, preferably at least 0.5 wt %, more preferably at least 0.75 wt % and, especially, at least 1 wt % form modifying material. Preferably, the amount of the form modifying material, for example coating material, is less than 5 wt % or less than 3 wt %.

Preferably, the weight of the excipients divided by the weight of the separate form modifying material, for example, coating material (which in the comparison is not to be regarded as an excipient) is in the range 0.02 to 0.1.

Step (iv) of the method may comprise extruding the precursor composition and subsequent treatment, suitably to produce elongated strands or threads. An extrusion-spheronisation technique may be used. The extruder may comprise extrusion orifices with a diameter between 0.001 mm and 10 mm, more preferably between 0.01 mm and 5 mm or between 0.1 mm and 2 mm, most preferably between 0.5 mm and 1.5 mm.

In step (iv), the method may comprise spheronisation or pelletising by using a spheroniser. The speed of the spheroniser may be at least 500 rpm, more preferably at least 1000 rpm and most preferably at least 1500 rpm. The spheronisation speed is preferably less than 5000 rpm, more preferably less than 4500 rpm, and most preferably less than 400 rpm.

In a step (v), the method may comprise drying of the pellets comprising the medicinal composition by placing under vacuum for a predetermined time. The drying step may be carried out at ambient temperature or, optionally, at an elevated temperature. It is preferably carried out under vacuum and at a temperature of between 20° C.-60° C., more preferably, at a temperature of between 25° C.-50° C.

The medicinal composition produced may comprise pellets or granules with an average diameter of between 10 μm and 5000 μm, more preferably between 100 μm and 4000 μm or between 250 μm and 3000 μm and most preferably between 500 μm and 2000 μm.

The method may include a step (referred to as step (vi)) which comprises associating a multiplicity of pellets comprising said medicinal composition with, for example arranging a multiplicity of pellets comprising said medicinal composition in, a material which is gastric resistant. Thus, enteric coated capsules which are gastric resistant are suitably produced. For example, the pellets may be packed or dry filled, into capsules coated in a gastric resistant gel.

Capsule may be selected from hard gelatin and hard hydroxypropyl methylcellulose or hypromellose (HPMC). The capsules may be made from materials whose dissolution is pH independent, and that are suitable for enteric coating. These may include starch capsules such as those manufactured from potato starch. The capsules may be manufactured from polyvinyl acetate (PVA). The said hard capsules are preferably suitable for controlled release of the active components in the medicinal composition. Preferably they may be coated with an enteric barrier that inhibit dispersal and/or dissolution In gastric acids in the stomach. Preferably the enteric coating is design to disintegrate and disperse in the relatively high pH conditions of the intestinal tract.

Most preferably, the medicinal composition may be dry filled into capsules that are made from acid resistant materials such as Eudracap™ enteric capsules.

Steps (i) to (vi) may be undertaken consecutively starting with step (i) and moving to step (vi).

According to a second aspect of the invention, there is provided a medicinal composition produced in the method of the first aspect.

According to a third aspect of the invention, there is provided a medicinal composition comprising pellets comprising an extract of cannabis plant material and a first excipient.

An extract of cannabis plant material may be as described in the first aspect. Said extract may include one or more purified cannabinoids, for example including THC and/or CBD and terpenes which have been extracted from a biomass from the cannabaceae plant family, for example from Cannabis sativa. Preferably, at least 90 wt %, more preferably at least 95 wt %, especially at least 98 wt % of said extract comprises a material which is naturally occurring in said cannabis plant material and has been extracted therefrom.

Said extract preferably includes multiple different compounds. It preferably includes at least one of THC or CBD. It preferably includes one or more, preferably a multiplicity, of terpenes. Set extract may include at least three, preferably at least six, more preferably at least ten different compounds. For example, it may be a broad-spectrum extract from Cannabis sativa.

Advantageously, it is found that, despite the fact the extract may include multiple different compounds, having multiple different solubilities, the medicinal composition is able to release components of the extract in a controlled manner which allows a patient to receive a therapeutic benefit, from a synergistic effect arising from multiple different active ingredients included in the extract.

The extract may be a broad-spectrum distillate derived, for example, by distillation of a full spectrum extract, trichome powder isolated from botanical biomass using mechanical means.

Said extract is preferably a liquid at atmospheric pressure and 25° C.

Said medicinal composition may include a residue of a liquid vehicle as described in the first aspect. The liquid vehicle may comprise a polyethylene glycol (PEG), a propylene glycol (PG), a polysorbate, a carboxylic acid, a mono-, di- and/or triglyceride, and/or an organic compound comprising a hydroxyl group, a carboxyl group and/or an ester group. The liquid vehicle may comprise a polysorbate. More specifically, the liquid vehicle may be a polyethylene ester with a chain of ethylene oxide units, sorbitol (or glucitol) and a primary fatty acid such as oleic acid. The liquid vehicle may have an average molecular weight between 200 g/mol and 10,000 g/mol, preferably in the range 750 g/mol to 2,500 g/mol and more preferably in the range 1,000 g/mol to 1,500 g/mol (1.0-1.5 KDalton).

In the medicinal composition, the weight of said extract may represent at least 5 wt %, preferably at least 10 wt %, more preferably at least 12 wt % (and suitably less than 25 wt %) of the total weight of the medicinal composition.

Preferably, the first excipient is as described in the first aspect. The first excipient may comprise a cellulose, a silicate, a phosphate and/or a material with a large BET (Brunaur, Emmet and Teller) specific surface area The material may have a BET specific surface area of at least 10 m²/g, at least 50 m²/g, at least 100 m²/g or at least 200 m2/g. When said first excipient comprises a cellulose, it may comprise a cellulose powder. Said cellulose may comprise microcrystalline cellulose (MCC), and more preferably MCC powder. The cellulose powder preferably comprises granules with a mean diameter of between 1 μm and 500 μm, more preferably between 10 μm and 250 μm or between 20 μm and 100 μm. When said first excipient comprises a silicate and/or a phosphate it may comprise magnesium aluminometasilicate or calcium phosphate. A calcium phosphate may be a dibasic calcium phosphate. A silicate and/or a phosphate described may have a BET specific surface area as described for said first excipient.

Said medicinal composition may include a second excipient, wherein said first and second excipients are different. Preferably, the second excipient is as described in the first aspect. The second excipient may comprise a cellulose, a silicate, a phosphate and/or a material with a large BET specific surface area. The material may have a BET specific surface area of at least 10 m²/g, at least 50 m²/g. most preferably at least 200 m²/g. When said second excipient comprises a cellulose, it may comprise a cellulose powder. Said cellulose may comprise microcrystalline cellulose (MCC), and more preferably MCC powder. The cellulose powder preferably comprises granules with a mean diameter of between 1 μm and 500 μm, more preferably between 10 μm and 250 μm or between 20 μm and 100 μm. When said second excipient comprises a silicate and/or a phosphate it may comprise magnesium aluminometasilicate or calcium phosphate. A calcium phosphate may be a dibasic calcium phosphate. A silicate and/or a phosphate described may have a BET specific surface area as described for said second excipient.

Said medicinal composition may include a third excipient which may be a disintegrant. The disintegrant may comprise alginate, chitin, chitosan, or a pharmaceutically acceptable salt thereof. The third excipient may comprise a starch- or cellulose-based excipient, or a pharmaceutically acceptable salt thereof, such as corn starch, partially pregelatinized starch, microcrystalline cellulose, and low-substituted hydroxypropyl cellulose. The third excipient is preferably a superdisintegrant. It may comprise starch glycolate, polyvinylpolypyrrolidone (PVPP), croscarmellose, chitin-silica, chitosan-silica, indion 414, mucilage of Plantago ovate, or a pharmaceutically acceptable salt thereof. The salt may comprise a sodium salt. The third excipient may comprise sodium starch glycolate, croscarmellose sodium or sodium alginate.

Said medicinal composition may include a fourth excipient which may comprise a retarding agent. The fourth excipient may be a polymer arranged to sustainably release the extract. The polymer may be a polymer based on a cellulose. For instance, the polymer may be hydroxypropyl methylcellulose (HPMC) or ethyl cellulose. The polymer may be a copolymer arranged to sustainably release the extract. The copolymer may be a copolymer of ethyl acrylate, methyl methacrylate and/or trimethylammonioethyl methacrylate chloride. Preferably, the copolymer is a copolymer of ethyl acrylate, methyl methacrylate and trimethylammonioethyl methacrylate chloride. The molar ratio of ethyl acrylate to methyl methacrylate may be between 1:100 and 100:1, more preferably between 1:2.5 and 1:1.5. The molar ratio of ethyl acrylate to trimethylammonioethyl methacrylate chloride may be between 1:0.001 and 1:10, more preferably between 1:0.06 and 1:0.5.

The sum of the wt % of excipients, for example, said first, second, third and/or fourth excipients in said medicinal composition may be at least 40 wt %, preferably at least 50 wt %. The sum may be less than 85 wt % or less than 80 wt %.

Preferably, the form modifying material, for example coating material, is an inert material. Preferably, the form modifying material comprises a powder. The form modifying material may have a BET specific surface area of at least 10 m²/g, at least 50 m²/g, or at least 200 m²/g. The form modifying material may comprise silica, magnesium aluminometasilicate or calcium phosphate. The silica may comprise fumed silica. The form modifying material, for example said coating material, is preferably a silica, for example a fumed silica.

Preferably, the amount of form modifying material, for example coating material, used is sufficient to cause the medicinal composition to comprise at least 0.25 wt % form modifying material, preferably at least 0.5 wt %, more preferably at least 0.75 wt % and, especially, at least 1 wt % form modifying material. Preferably, the amount of the form modifying material is less than 5 wt % or less than 3 wt %.

Preferably, the weight of the excipients divided by the weight of the separate form modifying material, for example coating material (which in the comparison is not to be regarded as an excipient), is in the range 0.02 to 0.1.

The medicinal composition produced may comprise pellets or granules with an average diameter of between 10 μm and 5000 μm, more preferably between 100 μm and 4000 μm or between 250 μm and 3000 μm and most preferably between 500 μm and 2000 μm.

Said medicinal composition may comprise a multiplicity of pellets comprising said medicinal composition in a material which is gastric resistant.

According to a fourth aspect of the invention, there is provided the use of a medical composition made as described in the first aspect or being as described in the second or third aspects for the treatment of a disease or disorder. In the use, preferably, the extract is predominantly released downstream of the stomach and/or in the small intestine.

According to a fifth aspect, there is provided a method of treating a disease or disorder which comprises administering to a patient a medical composition made as described in the first aspect or being as described in the second or third aspects for the treatment of a disease or disorder. In the method, preferably, the extract is predominantly released downstream of the stomach and/or in the small intestine.

The inventions of the fourth or fifth aspects suitably involve predominant release of the extract downstream of a patient's stomach and/or predominantly in the small intestine. Suitably, the wt % of the extract released downstream of the stomach and/or in the small intestine divided by the wt % of extract released in the stomach is at least 2, preferably at least 5 and, more preferably, at least 10.

Preferred embodiments of the invention may address the problems highlighted in the introduction because:
Administration of controlled, sustained-release ingested composition provides more precise, thus safer, dose management by both patient and prescribing clinician.
The elimination of gastric acids hydrolysis of the expensive actives content of the composition means that lower dose sizes are needed, hence lower costs.
A more direct route of administration to the systemic circulation system means better and more precise dosing regime and management.
The dangers of direct inhalation of substances to the lungs via smoking or vaping are fully understood.
The precisely measured dose sizes eliminate risks of accidental overdosing inherent in sublingual drops and nasal sprays and minimise risk of interactions with other medications.

Any feature or any aspect of any invention or embodiment described herein may be combined with any feature of any aspect of any other invention or embodiment described herein mutatis mutandis.

Specific examples of the present invention will now be described, by way of example, with reference to the following figures, in which:

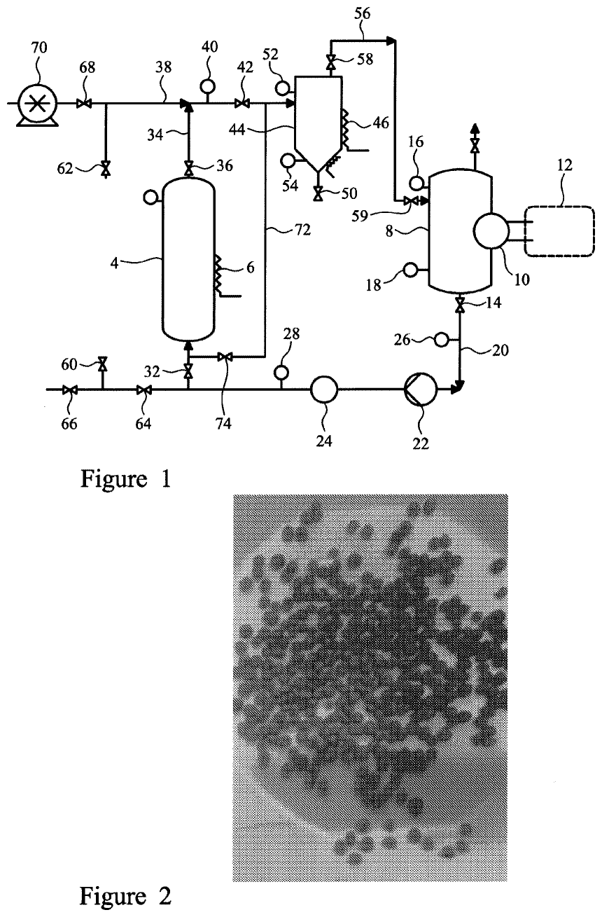

FIG. 1 is a schematic representation of apparatus for extraction from a biomass;

FIG. 2 is a photograph of CBD-containing spheronised pellets;

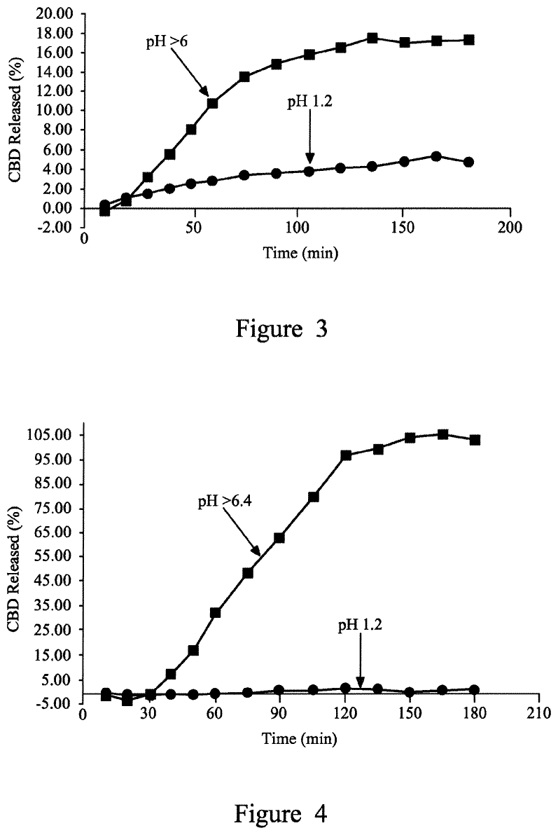

FIG. 3 is a graph including comparative release profiles of fast release capsules comprising a commercial product;

FIG. 4 is a graph including comparative release profiles of fast release capsules comprising the formulation of Example 7;

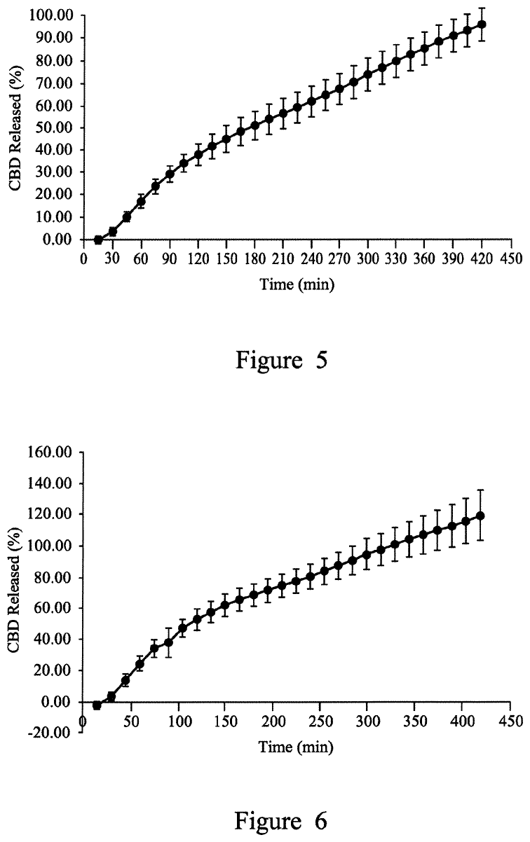

FIG. 5 is a graph showing the release profile of slow-release capsules comprising the formulation of Example 5;

FIG. 6 is a graph showing the CBD release profile of the formulation of Example 10;

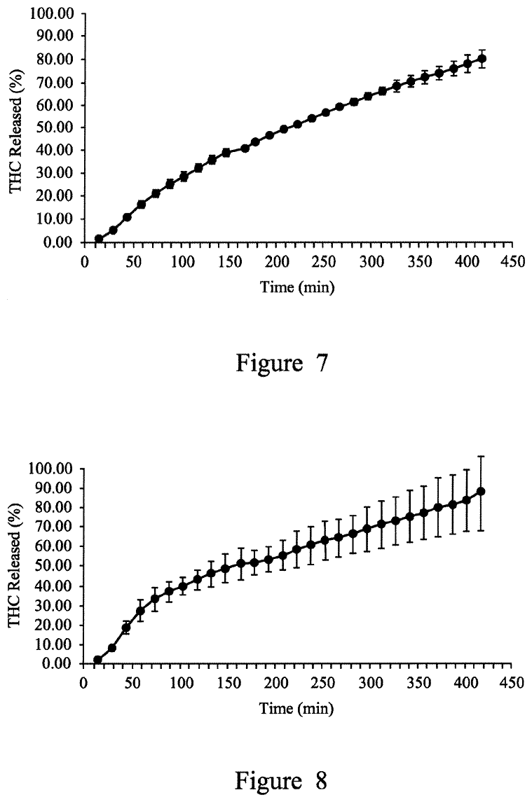

FIG. 7 is a graph showing the THC release profile of the formulation of Example 11;

FIG. 8 is a graph showing the THC release profile of the formulation of Example 12;

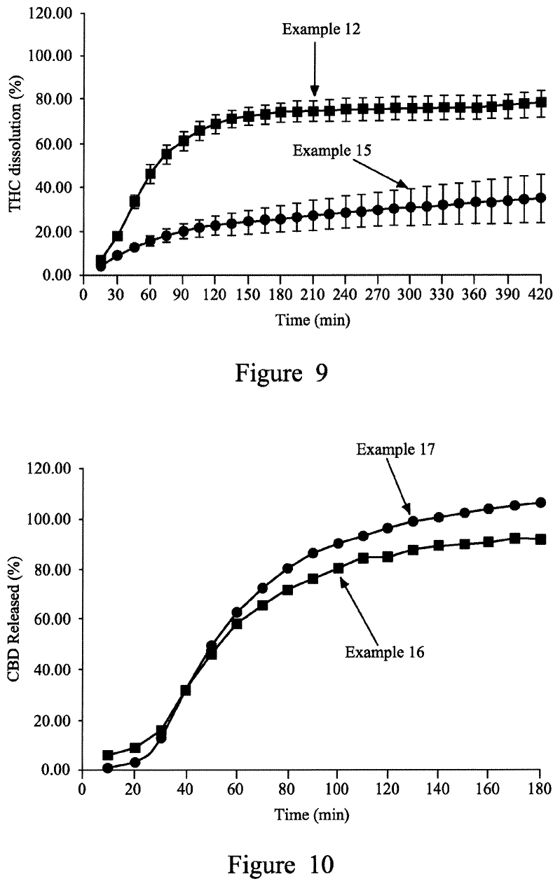

FIG. 9 is a graph including comparative release profiles of the formulation of Example 12 and raw Trichome powder as described in Example 15;

FIG. 10 is a graph illustrating specified fast release formulations;

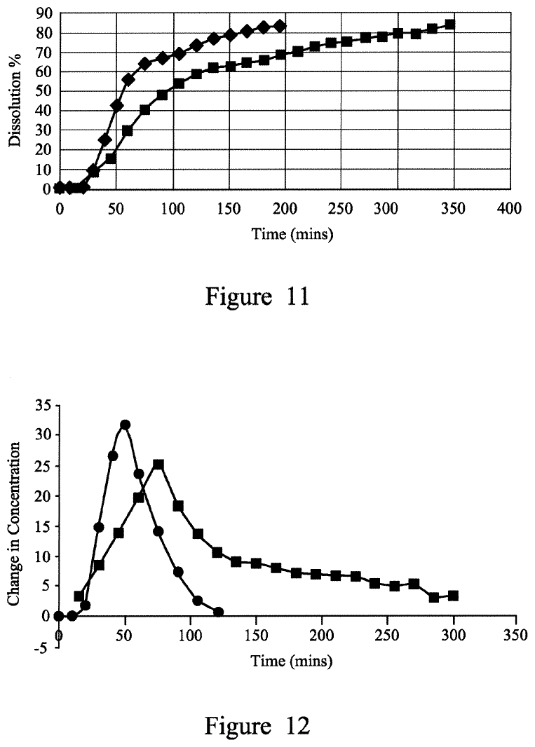

FIG. 11 is a graph illustrating slow and fast THC dissolution rates:

FIG. 12 is a graph comparing the change in concentration (μmole) of fast release vs. slow release formulations of whole spectrum THC extract;

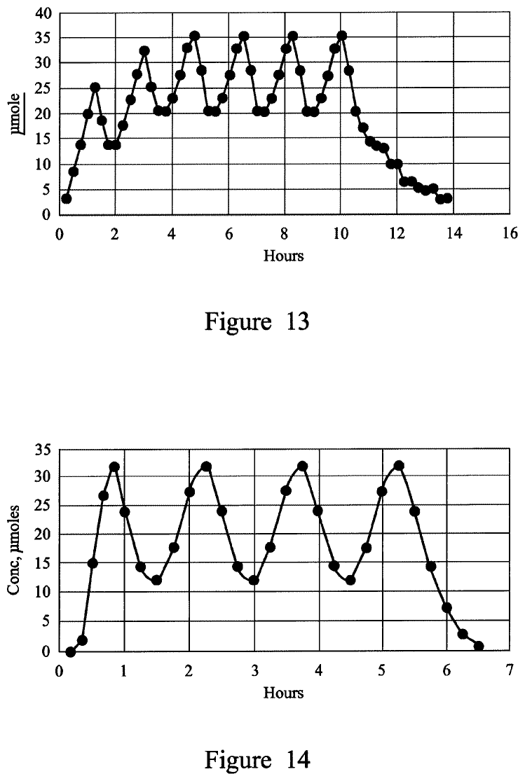

FIG. 13 is a graph illustrating multi-dose sustained release of THC formulations; and

FIG. 14 is a graph illustrating multi-dose sustained release of a formulation.

The following are referred to hereinafter:

HFC134a-refers to 1, 1,1,2-tetrafluoroethane.

Avicel PH 101 and Avicel PH-102 excipients manufactured by Dupont and used to improve powder flow and compressibility.

Capsule material A-EUDRACAP™ functional ready-to-fill, coated (HPMC) capsules that protect APIs and optimize their release profile.

Neusilin an amorphous magnesium aminometrasilicate excipient manufactured by Fuji Industries, used for as an oil absorbent to aid in tabletting manufacture.

Trichome powder are fibrous powders containing cannabinoids and terpenes isolated fom cannabis trichomes.

In general terms, poorly water-soluble, liquid extracts prepared as described can be formulated, using the processes described herein, to define dosage forms which are able to release the active ingredients, for example, substantially full-spectrum extracts comprising cannabinoids and terpenes, at a generally constant and consistent rate over a relatively long, defined period of time. Dosage forms may be prepared in the form of capsules which include high levels of CBD on the one hand or which include high levels of THC on the other hand, as may be desired.

### General Process and Apparatus for Producing Extracts

Apparatus 2 for extraction of a biomass comprises an extraction vessel 4 for containing a biomass to be extracted. Electrical heating tape or an electrical heating blanket (shown schematically and referenced 6) is provided around and in contact with the vessel 4 for maintaining the temperature of the wall of vessel 4 and, consequently, the contents therein, within the desired limits.

Upstream of vessel 4 is a solvent recycling vessel 8 which is fitted with a cooling coil (shown schematically by reference numeral 10) which communicates with an external refrigeration unit 12. Operation of unit 12 is arranged to cool (and thereby liquefy) solvent in the vessel 8. A pressure sensor 16 and a temperature sensor 18 are provided for monitoring temperature and pressure within vessel 8. A pipe 20 communicates with an outlet of vessel 8 and is arranged to transport solvent to a solvent pump 22. Valve 14 is provided adjacent to the outlet of vessel 8 for controlled passage of solvent from vessel 8 into pipe 20.

The solvent pump 22 is arranged to pump liquid between vessel 8 and vessel 4 via an electrically powered heat exchanger 24. A temperature sensor 26 is arranged to monitor temperature of solvent in pipe 20.

The heat exchanger 24 is arranged to increase the temperature of solvent flowing through it very rapidly. For example, it is suitably arranged to increase temperature of solvent between its inlet and outlet from as low as −30° C. to as high as 30° C. in less than 1 minute. This allows the temperature of solvent introduced into extraction vessel 4 to be very rapidly changed in a step-wise manner rather than temperature of solvent being changed linearly over an extended period of time. This is found to significantly affect speed, efficiency and controllability of extraction processes using the apparatus and facilitates production of higher quality extracts.

The heat exchanger may have a working length between its inlet and outlet of between 800 mm and 1400 mm. The pipe with which the heat exchanger is associated may have an internal diameter of between 6 mm and 20 mm. The heat exchanger is electrically operated, since such operation has been found to be able to produce a sufficiently rapid temperature increase of solvent passing through. Said heat exchanger may have a heater resistance of about 1000 per metre; it may be operated at 240V ac.

Downstream of the heat exchanger 24 is a temperature sensor 28 for monitoring temperature of solvent in pipe 30, after passage through the heat exchanger. Pipe 30 is arranged to transport solvent into vessel 4 via a valve 32. Vessel 4 may have a volume of 3L-5L for containing biomass to be extracted.

A pipe 34, in which valve 36 is arranged, is provided downstream of vessel 4. Pipe 34 communicates with a pipe 38 which is arranged to transport solvent via temperature sensor 40 and valve 42 to an evaporator vessel 44. This vessel 44 is wrapped with electrical heating tape or an electric heating blanket (shown schematically and referenced 46). Vessel 44 includes a pressure sensor 52 and a temperature sensor 54. It also includes an outlet and associated valve 50 via which extract can be drawn off from the apparatus 2.

Valve 36 incorporates a pressure sensor so that a pressure differential between vessel 4 and vessel 44 is maintained. This mechanism to ensure that the pressure within vessel 4 is always kept above the vapour pressure so that the solvent medium within vessel 44 is maintained in a liquid state.

A pipe 56 is arranged to transport solvent back to vessel 8 via valves 58, 59.

Also illustrated in FIG. 1 are valves 60, 62, 64, 66 and 68 which may be associated with and/or arranged to control passage of solvent to/from a second extraction vessel (not shown) which may be as described for vessel 4 and may be positioned between valves 60, 62.

The apparatus also includes a vacuum pump 70 for facilitating vacuum aided evaporation of solvent in evaporator vessel 44. In addition, the apparatus includes a shunt pipe 72 and associated valve 74 for returning solvent from pipe 38 to extraction vessel 4, if required at the end of the extraction run.

In general terms, the apparatus 2 may be operated as follows.

A biomass to be extracted is packed into extraction vessel 4. Then, vacuum pump 70 is operated to remove air from the apparatus. With HFC134a in vessel 8, refrigeration unit 12 is used to maintain the solvent at a suitable temperature and pressure in the liquid state, with the state of the solvent being monitored by pressure and temperature sensors 16, 18.

The solvent is pumped from vessel 8, in pipe 20, by pump 22 and the temperature of the solvent is monitored by temperature sensor 26. The solvent then passes into and through the heat exchanger 24. The heat exchanger 24 is operated to rapidly increase the temperature of the solvent as may be required during extraction of biomass. Typically, the solvent enters the heat exchanger at a temperature in the range −30° C. to −10° C. In one embodiment, the heat exchanger may be operated simply to maintain this low temperature in which case the temperature of solvent measured by temperature sensor 28 may be the same as that measured by sensor 26. Such a low temperature may be used to extract the most soluble components in the biomass. Alternatively, or additionally, the heat exchanger may be operated to very rapidly increase the temperature of solvent passing through it. For example, the heat exchanger may be operated to produce a temperature rise of 10 to 50° C. in less than 1 minute. In one embodiment, the heat exchanger may be operable to increase the solvent temperature between 1° and 50° C. per metre of heater exchanger in less than 1 minute or even less than 20 seconds. In one embodiment the extraction may be carried out under isocratic temperature conditions.

Vessel 4 temperature is either a step-wise gradient, or a steady state temperature that may be pre-set somewhere between 10° C. and 30° C. The temperature in vessel 44 is maintained in the steady state at predetermined anywhere between 2° and 30° C. Similarly, temperature in vessel 8 is suitably at a steady state as described.

From the heat exchanger 24, the solvent is pumped via pipe 30 and valve 32 into extraction vessel 4. It will be appreciated that the temperature of the solvent entering vessel 4 affects what is extracted from the biomass. At lower solvent temperatures, only the most soluble constituents will be extracted from the biomass; as the solvent temperature increases, a more complex mixture of components, which will be more concentrated in less soluble constituents, will be extracted. Advantageously, it is found that the ability to rapidly change the temperature of the solvent can be manipulated to produce higher quality extracts in contrast to situations wherein solvent temperature is changed more slowly over a relatively prolonged period which is the case with the apparatus of GB2393720B.

Solvent may be passed through the biomass at a rate of 30-70 L/hour or more ideally 40-60 L/hour. The solvent and entrained constituents extracted from the biomass exit vessel 4 at its upper end and pass via valves 36, 42 and pipes 34, 38 to the evaporator vessel 44. The temperature (and pressure) of the liquid in vessel 4 are monitored by temperature and pressure sensors 52, 54 and temperature adjusted, as necessary, by operation of electrical heating blanket/tape 46. Consequently, the HFC134a solvent is evaporated and transferred via valve 58, pipe 56 and valve 59 back to the recycling vessel 8. Valve 36 is electronically controlled by a pressure signal so that it opens and closes whilst keeping the pressure within vessel 8 at a predetermined level above that of the vapour pressure of the solvent. This ensures the liquid status of the solvent within the vessel 8 is maintained.

The vessel 8 is cooled by refrigeration unit 12 so as to maintain sufficient vapour pressure differential between vessels 44 and 8 so that simultaneous evaporation of the HFC134a solvent in vessel 44 and re-condensing in vessel 8 occurs continuously. This is made possible by the ability to accurately and rapidly monitor temperature and pressure in vessels 44 and 8 and the ability to rapidly adjust the temperature of liquid in vessel via electrical heating blanket/tape 46.

After evaporation of HFC134a solvent, extract remains in the evaporation vessel 44 from which it can be drawn off via valve 50. The selectivity of this process, inherent in the choice of solvent as well as the optimum extraction conditions, means that the required molecules, including the cannabinoids and terpenes, are efficiently extracted whilst most of the unwanted molecules, which mostly are plant waxes and heavy molecular weight polyphenols, are not extracted. The extract obtained via this process does not require further downstream purification steps such as winterisation and/or chromatography. Therefore, the extract obtained may be described as a primary and full spectrum with respect to the desired molecules which are primarily cannabinoids and terpenes.

Advantageously, the apparatus does not need a compressor to re-liquefy the solvent and this fact enables the apparatus to be operated according to GMP thereby allowing the extracts to be classified as Botanical Drug Substances and/or to be authorised for use as pharmaceuticals.

The biomass used in the apparatus is suitably Cannabis sativa, although the apparatus may be used to produce full spectrum extracts from other members of the cannabaceae plant family such as cannabis indica and ruderalis.

The following examples further illustrate the invention.

### Example 1-Preparation of Cannabis sativa for Extraction

Cannabis sativa biomass was dried to a water content of 20 wt %, milled to an average particle size of 1-3 mm and decarboxylated using standard thermal methods.

### Example 2-Extraction Using the Apparatus of FIG. 1

Decarboxylated biomass (ca. 90 g) was packed into a stainless-steel extraction vessel 4 in the form of a column having dimensions of 32 mm/500 mm, compacted using mechanical means into a tightly packed bed and assembled with the other parts of the apparatus as shown in FIG. 1. With valves 64, 68, 62 and 7 closed, all the constituent elements of the apparatus were evacuated using the vacuum pump 70. With valves 14 and 59 closed, the solvent vessel 8 was charged with 4 Kg HFC134a via valve 7 and cooled down and maintained within the temperature range of −30° C. and −70° C. throughout the subsequent extraction operation. With valves 14, 32, 36 and 42 opened, the solvent pump 22 was started so that cold HFC134a passed via the heat exchanger 24 into the extraction vessel 4 in an up-flow mode so that mass transfer of some constituent compounds from the biomass into the solvent was caused to occur. The now “rich” HFC134a solution containing the extracted compounds was continuously directed via valves 36 and 42 into the evaporator 44, where it was heated using the electrically operated jacket causing evaporation of the solvent. The resulting pressure differential between the evaporator vessel 44 and the solvent vessel 8 caused the solvent vapour to flow into the solvent 8 where it was continuously reliquefied prior to recycling through the biomass whilst the extracted compounds were collected in the evaporator vessel. A steady state was maintained as follows for 1.5 hours, the operating parameters, with reference to FIG. 1, being as follows:
temperature sensor 28=15° C.-25° C.
temperature sensor 54=15° C.-25° C.; pressure sensor 52=4.5 bar-5.5 bar
temperature sensor 18=−30° C.-60° C.; pressure sensor 16=1.0 bar-2.5 bar.

At the end of the extraction, the solvent pump 22 was switched off, valve 14 closed and valve 74 opened. Temperature and pressure steady state were maintained until all the HFC134a was transferred via the evaporator vessel into the solvent storage vessel. The extracted product collected in the evaporator vessel was harvested, weighed and analysed by gradient HPLC.

### Example 3

A THC rich biomass was decarboxylated by placing the biomass in an oven at 90° C. for 2 hours and then treated as described in Example 2.

### Example 4

A CBD rich biomass was decarboxylated by placing the biomass in an oven at 90° C. for 2 hours and then treated as described in Example 2.

Extracts produced by treating both THC and CBD rich biomasses using HFC134a and the apparatus described had an oily consistency and were light yellow/light brown in colour. The extracts are used in controlled release formulations as hereinafter described.

### Example 5-Preparation of a Pellet Containing Liquid CBD-Containing Extract

Pellets were prepared by mixing CBD extract (80%) in Tween 80 based on the amount listed in the table below.

| | | | | | Avicel | | | | | | | | |
|---|---|---|---|---|---|---|---|---|---|---|---|---|---|
| | | | | | PH 102 or | | | | | | | Total | |
| EXAMPLE 5 | CBD | | Tween 80 | | PH 101 | | Neusilin | | Aerosil | | water | weight | |
| Amount | 2.5 | g | 6 | g | 6 | g | 4 | g | 0.5 | g | 2 ml | 19 | g |
| Each capsule | 50 | mg | 120 | mg | 120 | mg | 80 | mg | 8 | mg | | 380 | mg |

The mixing (known as liquid medication) was carried out using a pestle and mortar. After thorough mixing, Avicel PH-102 (Avicel PH 101 can also be used) was mixed into the admixture to make sure the wet liquid medication was absorbed by the carrier without leaving any residue in the mortar. Then other components (except Aerosil) were added and mixed for 5 minutes to achieve a uniform distribution of liquid medication in the admixture. Aerosil 300 was then added into the admixture and further mixed for 5 minutes before an extrusion-spheronization process, using a Caleva Multilab (supplied by Caleva Process Solutions Ltd., UK). As soon as the mixture was ready, approximately 2 ml of water was added slowly and mixed thoroughly to achieve desirable plasticity for a subsequent extrusion step. Applicant's studies have determined that water content may be adjusted to obtain extrudates with optimal plasticity. The amount of water may be varied dependant on the quantity and type of the other excipients used in the formulation.

The wet mass produced by the mixing step was extruded using a screw type spheronizer at a speed of between 100-120 rpm. The subsequent spheronization step was carried out at a constant rotation rate of 3000 rpm. The resulting spherical pellets were oven dried overnight at a constant temperature of 50° C. The dried spherical pellets were packed into capsules (eg comprising Capsule material A), coated in gastric resistant gels and dried at room temperature or in an oven at up to 50° C. FIG. 2 shows dried spherical pellets produced as described.

### Example 6—General Procedure for Dissolution Testing

Coated capsules containing formulations to be assessed were subjected to dissolution tests as follows: All dissolution tests were carried out using USP basket method (708-DS Dissolution Apparatus & Cary 60 UV-Vis, Agilent Technologies, USA). Formulations comprising pellets in capsules were suspended in deionised water/SDS buffer solution at a) pH 1.0-1.2 and b) pH 7-7.5 of the test solution in the above apparatus under constant conditions of 900 ml of deionised water containing 1% SDS (pH around 6.2) or HCl solution (pH 1.2) as dissolution medium, paddle agitation of 75 rpm, and temperature of 37.3+0.5° C. The dissolution medium was either HCl buffer solution of pH 1.2 or distilled water containing 1% SDS. Absorbance (at 278 nm—the predetermined maximum absorbance peak) was taken at different time intervals. In order to allow a correlation between absorbance detected and the mg of extract released from a formulation to be estimated, an assay of the extract was undertaken, wherein 20 mg of extract was completely dissolved in 2 ml of ethanol, then made up to 100 ml with 1% SDS solution and its absorbance at 278 λ measured (0.979). This value was then used to calibrate the absorbance values recorded during the dissolution testing.

### Example 7-Preparation of Alternative Pellet Containing Liquid CBD

The ingredients detailed in the table below were mixed following the procedure of Example 5.

| | | | | | | | | Total weight |
|---|---|---|---|---|---|---|---|---|
| Example | CBD/ | Tween80/ | Avicel/ | Neusilin/ | Primolgel/ | Aerosil/ | Water/ | after drying/ |
| No | g | g | g | g | g | g | mL | g |
| 7 | 0.9908 | 6 | 6 | 4 | 0.6 | 0.4 | 2.13 | 14.15 |

### Example 8-Analysis of Fast-Release CBD Formulations and Comparison with Commercially-Available Capsules

Samples from Example 7 were tested as described in Example 6 and were found to exhibit fast and controlled release profile.

In order to assess the commercial viability of the formulation of Example 7, the formulation was compared directly with a commercially available product. To facilitate this, six capsules were prepared by loading 358 mg of the formulation of Example 7 into respective capsules A and the capsules were dip coated into a solution of cellulose acetate phthalate solution (260 mg in 100 mL). The dipping process was carried out to provide acid resistance ensuring the capsules pass through the stomach intact. Each capsule was weighed before and after coating to ensure uniformity. The capsules were then tested against an enteric coated commercial product of comparable concentration. Both the commercial product and capsules comprising the formulation of Example 7 were tested over a pH range comparable to the stomach and the lower intestine. The results showed that capsules comprising the formulation of Example 7 released the CBD extract at a much faster and more uniform rate than the commercial product (compare FIG. 4 which provides results for capsules comprising the formulation of Example 7 to FIG. 3 which shows results for the commercial product). Additionally, capsules comprising the formulation of Example maintained structural integrity much better at the lower pH of the stomach.

FIG. 3 also shows that under simulated gastric conditions of pH1.2, the commercial product showed a higher release than for capsules comprising the formulation of Example 7 shown in FIG. 4, whilst the rate of release under simulated small intestine conditions (pH>6 is much lower than the rate for capsules comprising the formulation of Example 7-18% release compared to around 100% release in FIG. 4.

### Example 9-Slow Release Formulations

Primojel, referred to in Examples 7 and 8 was found to take up water rapidly, and it was found that the rapid absorption of water allows high penetration and swelling, promoting faster breakdown of the pellets. The formulation of Example 7 may be compared with the formulation of Example 5 which is found to be capable of delivering a much slower release profile, by omitting sodium starch glycolate (Primojel). To illustrate, referring to FIG. 5, over 95% release took 390 minutes for the slow-release formulation compared with 135 minutes for the capsules comprising the formulation of Example 7 (see FIG. 4).

### Example 10

To further slow the release of CBD, Eudragit was added to formulation for Example 10 as detailed below.

| | | | | | | | | | Total weight |
|---|---|---|---|---|---|---|---|---|---|
| Example | CBD/ | Tween 80/ | Avicel/ | Neusilin/ | Primolgel/ | Aerosil/ | Eudragit/ | Water/ | after drying/ |
| No | g | g | g | g | g | g | g | mL | g |
| 10 | 2.51 | 7 | 6 | 3 | 0 | 0.5 | 2 | 2.0 | 15.97 |

Inclusion of Eudragit resulted in a much more elastic extrudate requiring much longer and slower spheronization. Dissolution results are provided in FIG. 6 which shows extended sustained release, over 420 minutes, which may be advantageous in treatment of sleep deprivation, wherein patients require less frequent dosing.

### Examples 11 and 12-Preparation of THC Formulations

THC formulations were prepared from two sources: one an extract solvent extracted using R134a using apparatus described in FIG. 1 and the other a powder derived from the trichomes of the cannabis plant (herein “Trichome powder”). The R134a extract was an oily reside containing 80% THC by high performance liquid chromatography (HPLC) and had already been decarboxylated. The Trichome powder was a solid containing only 40% THC and was decarboxylated prior to use by heating in an oven at 100° C. overnight.

Solubility analysis was undertaken and it was found that the extract was very soluble in polyoxyethylene (20) sorbitan monooleate (Tween 80) and soluble in 1% sodium dodecyl sulfate (SDS) solution. The 1% SDS solution was deemed a suitable substrate for dissolution studies as it has a pH analogous to the small intestine.

Initial UV-vis testing observed the max absorption peak was observed at 279 λ in 1% (SDS) solution. As described in Example 5, the pellets were prepared by first dissolving the extract in Tween 80 in a pestle and mortar. Once the extract was fully dissolved, the other excipients were added in the weights outlined in the tables below (table 2 and 3). The materials were then ground together for 5 minutes until homogenous. Once fully combined, a defined volume of water was added dropwise to the paste under constant mixing until the desired plasticity required for successful extrusion was achieved. A single screw extruder was set to a constant screw speed of 100-120 rpm. Once extruded, the formulation was loaded into a spheronizer at a rotation speed of 3000 rpm for times dependant on the plasticity of the extrudate. The formed pellets were then dried under vacuum desiccation overnight to remove the water.

| | | | | | | | Total weight |
|---|---|---|---|---|---|---|---|
| Formulation | THC/ | Tween 80/ | Avicel/ | Neusilin/ | Aerosil/ | Water/ | after drying/ |
| designation | g | g | g | g | g | mL | g |
| Example 11 | 2.5 | 6 | 6 | 4 | 0.5 | 2.7 | 16.38 |
| Example 12 | 2.5 | 5 | 6 | 4 | 0.5 | 5.11 | |

### Example 13-Analysis of THC Formulations of Examples 11 and 12

In order to allow a correlation between absorbance detected and the mg of extract released from the formulations to be estimated an assay of the extract was undertaken. 20 mg of TFC extract was used in Example 11, completely dissolved in 2 mL of ethanol then made up to 100 mL with 1% SDS solution and its absorbance at 279 λ measured giving a value of 1.013. This value was then used to calibrate the absorbance values recorded during the dissolution testing for extract derived formulation. The same process was performed on the Trichome powder formulation of Example 12 giving a value of 1.202. To ensure the formulation did not exceed the solubility of CBD extract in the volume of 1% SDS solution used in the dissolution study, a solubility screen was undertaken.

### Example 14-Slow-Release Formulations

When tested using dissolution apparatus analysed by Ultraviolet-visible (UV-vis) spectroscopy, both THC formulations of Example 11 and 12 exhibited a good slow-release profile (FIG. 7) with the extract derived formulation of Example 11 yielding a more linear release. This could either be a result of the comparatively high water content in the powder formulation or impedance by other compounds found in the Trichome powder.

### Example 15

In order to test the effectiveness of the formulation process directly for THC, 50 mg of the raw decarboxylated Trichome powder was loaded into three uncoated capsules and three more loaded with 360 mg of the formulation of Example 12. These six capsules were then tested using dissolution apparatus analysed by Ultraviolet-visible (UV-vis) spectroscopy. The results showed efficacy of the pellet formulation with the Example 12 capsules releasing the THC much faster than the powder alone (FIG. 9). A visual inspection of the chambers showed a stark contrast between the two: with the formulation partially suspended in the solution while the powder was floating on the surface.

### Example 16 to 17

Products currently approved (licensed) in relation to CBD and/or THC include:
a) Epidiolex™—this includes an active ingredient which is pure CBD isolate;
b) Sativex™—this includes an active ingredient which is a 1:1 y weight mixture of purified CBD/THC isolates;
c) Dronabinol—this includes THC isolates or purified or broad-spectrum distillate containing primarily THC.

The following examples demonstrate the applicability of the process described herein and the dosage forms described for examples of active ingredients analogous to those of licensed products.

Formulations were prepared as described in Example 7 mutatis mutandis. Fast release formulations comprising 99% pure CBD isolate (for the Example 16 formulation) and a broad-spectrum distillate containing CBD as primary ingredient (for the Example 17 formulation) were prepared.

Results are provided in FIG. 10 which illustrates that both samples show very similar, linear, dissolution profiles, with most of the CBD released within 2 hours.

### Example 18 and 19

Slow (Example 18) and fast (Example 19) release formulations comprising broad spectrum THC extract were prepared as described in Example 7 mutatis mutandis and dissolution results at pH 7.4 are provided in FIG. 11. However, whereas graphs such as that in FIG. 11 shows the amount of CBD or THC which accumulates, when formulations are used medically, the CBD or THC will be metabolised, meaning that the CBD or THC will not simply accumulate but will tail off over time. This is illustrated in FIG. 12 which shows the change in concentration of the slow (Example 18) and fast (Example 19) release formulations.

### Example 20

Using formulations based on those described herein, simulated multi-dose sustained slow release formulations may be prepared. Such formulations may have a dose interval of 2 hours and may be used for chronic pain treatment.

### Example 21

Using formulations based on those described herein, simulated multi-dose sustained fast release formulations may be prepared. Such formulations may have a dose interval of 1.5 hours and may be used for chronic pain treatment.

The graphs of FIGS. 13 and 14 demonstrate multi dose administration, for example in cancer treatment, to provide a base line sustained pain relief.

The invention is not restricted to the details of the foregoing embodiment(s). The invention extends to any novel one, or any novel combination, of the features disclosed in this specification (Including any accompanying claims, abstract and drawings), or to any novel one, or any novel combination, of the steps of any method or process so disclosed.

1. A method of producing a medicinal composition comprising an extract of cannabis plant material, the method comprising:
(i) mixing, for example dissolving, an extract of cannabis plant material in a liquid vehicle to form a liquid mixture;
(ii) contacting the liquid mixture with a first excipient;
(iii) contacting the combination comprising the liquid mixture and said first excipient with a form modifying material, for example a coating material, to form a precursor composition; and
(iv) treating, for example pelletising, the precursor composition to form the medicinal composition.

2. A method according to claim 1, wherein said extract includes THC and CBD, and one or more terpenes which have been extracted from said plant material,
wherein at least 98 wt % of said extract comprises a material which is naturally occurring in said cannabis plant material and
wherein said extract is a liquid at atmospheric pressure and 25° C.

3. (canceled)

4. (canceled)

5. (canceled)

6. (canceled)

7. A method according to any preceding claim 1, wherein said the liquid vehicle is a non-ionic organic liquid selected from a polyethylene glycol (PEG), a propylene glycol (PG), a polysorbate, a carboxylic acid, a mono-, di- and/or triglyceride, and/or an organic compound comprising a hydroxyl group, a carboxyl group and/or an ester group.

8. A method according to claim 7, wherein the weight ratio of the extract to the liquid vehicle, defined as the weight of extract divided by weight of said liquid vehicle, is in the range 0.1 to 1, for example in the range 0.1 to 0.6.

9. A method according to claim 1, wherein, in the medicinal composition, the sum of the weights of the extract and said liquid vehicle represent 20-80 wt %, of the total weight of the medicinal composition; and/or in the medicinal composition, the weight of said liquid vehicle represents at least of the total weight of the medicinal composition.

10. A method according to claim 9, wherein, in the medicinal composition, the weight of said extract represents at least 5 wt % of the total weight of the medicinal composition.

11. A method according to claim 1, wherein the first excipient comprises a cellulose, a silicate, a phosphate and/or a material with a BET (Brunaur, Emmet and Teller) specific surface area of at least 10 m²/g.

12. A method according to claim 11, wherein said first excipient comprises microcrystalline cellulose, magnesium aluminometasilicate or calcium phosphate.

13. A method according to claim 1, wherein the method comprises contact with said first excipient and a second excipient, wherein said first and second excipients are different and the second excipient comprises a cellulose, a silicate, a phosphate and/or a material with a BET specific surface area of at least 10 m²/g.

14. A method according to claim 13, wherein the method comprises contact with said first excipient and/or said second excipient and a third excipient, wherein said third excipient is a disintegrant, for example selected from alginate, chitin, chitosan, a starch, starch glycolate, polyvinylpolypyrrolidone (PVPP), croscarmellose, chitin-silica, chitosan-silica, indion 414, mucilage of Plantago ovate, or a pharmaceutically acceptable salt thereof.

15. A method according to claim 14, wherein the method comprise contact with a fourth excipient which is a retarding agent, for example selected from a polymer based on a cellulose, a copolymer of ethyl acrylate, methyl methacrylate and/or trimethylammonioethyl methacrylate chloride.

16. A method according to claim 1, wherein the sub of the wt % of excipients.

17. A method according to claim 14, wherein the form modifying material is an inert material which has a BET specific surface area of at least 10 m²/g and is optionally selected from silica, magnesium aluminometasilicate and calcium phosphate.

18. A method according to claim 17, wherein the amount of form modifying material used is sufficient to cause the medicinal composition to comprise at least 0.25 wt % form modifying material; and/or the amount of the form modifying material is less than 5 wt %.

19. A method according to claim 16, wherein the wright of the excipients divided by the weight of the separate form modifying material is in the range 0.02 to 0.1.

20. A method according to claim 1, wherein:
step (iv) of the method comprises extruding the precursor composition and subsequent treatment, to produce elongated strands or threads; and/or
step (iv) comprises spheronisation or pelletising by using a spheroniser.

21. (canceled)

22. A method according to claim 20, wherein the medicinal composition produced comprises pellets or granules with an average diameter of between 250 μm and 3000 μm.

23. A method according to claim 1, the method include a step (referred to as step (vi)) which comprises associating a multiplicity of pellets comprising said medicinal composition with a material which is gastric resistant.

24. A medicinal composition produced in the method of claim 1, wherein:
said medicinal composition comprises pellets comprising an extract of cannabis plant material and a first excipient;
said extract includes THC and CBD and one or more terpenes which have been extracted from a cannabis plant arterial and wherein at least 98 wt % of said extract comprises a material which is naturally occurring in said cannabis plant material:
said medicinal composition include a residue of a liquid vehicle comprising a polyethylene glycol (PEG), a propylene glycol (PG), a polysorbate, a carboxylic acid, a mono-, di- and/or triglyceride, or an organic compound comprising a hydroxyl group, a carboxyl group or an ester group:
in the medicinal composition, the weight of said extract represents at least 10 wt % of the total weight of the medicinal composition:
said medicinal composition includes:
a first excipient comprising a cellulose, a silicate, or a phosphate;
a second excipient, wherein said first and second excipients are different and the second excipient comprises a cellulose, a silicate, or a phosphate;
a third excipient which is a disintegrant, selected from a starch- or cellulose-based excipient, or a pharmaceutically acceptable salt thereof;
a fourth excipient which comprises a retarding agent selected from a polymer based on a cellulose, a copolymer of ethyl acrylate, a methyl methacrylate and/or trimethylammonioethyl methacrylate chloride;
wherein the sum of the wt % of excipients in said medicinal composition is at least 40 wt %;
said composition includes a form modifying material having a BET specific surface area of at least 10 m²/g which comprises a silica, magnesium aluminometasilicate or calcium phosphate;
the amount of form modifying material is sufficient to cause the medicinal composition to comprise at least 0.25 wt % form modifying material;
the medicinal composition comprises pellets or granules with an average diameter of between 10 μm and 5000 μm; and
said composition comprises a multiplicity of pellets comprising said medicinal composition in a material which is gastric resistant.

25-38. (canceled)

39. A method according to claim 2, wherein:
said the liquid vehicle is a non-ionic organic liquid selected from a polyethylene glycol (PEG), a propylene glycol (PG), a polysorbate, a carboxylic acid, a mono-, di- and/or triglyceride and an organic compound comprising a hydroxyl group, a carboxyl group or an ester group;
the weight ratio of the extract to the liquid vehicle, defined as the weight of extract divided by weight of said liquid vehicle, is in the range 0.1 to 0.6;
in the medicinal composition, the weight of said extract represents at least 12 wt % of the total weight of the medicinal composition;
the first excipient comprises a cellulose, a silicate, a phosphate and/or a material with a BET (Brunaur, Emmet and Teller) specific surface area of at least 50 m²/g;
the method comprises contact with said first excipient and a second excipient, wherein said first and second excipients are different and the second excipient comprises a cellulose, a silicate, a phosphate and/or a material with a BET specific surface area of at least 50 m²/g;
the method comprises contact with said first excipient and said second excipient and a third excipient, wherein said third excipient is a disintegrant selected from alginate, chitin, chitosan, a starch, starch glycolate, polyvinylpolypyrrolidone (PVPP), croscarmellose, chitin-silica, chitosan-silica, indion 414, mucilage of Plantago ovate, or a pharmaceutically acceptable salt thereof;
the sum of the wt % of excipients in said medicinal composition is at least 40 wt %;
step (iv) of the method comprises extruding the precursor composition and subsequent treatment to produce elongated strands or threads or step (iv) comprises spheronisation or pelletising by using a spheroniser; and
the medicinal composition produced comprises pellets or granules with an average diameter of between 250 μm and 3000 μm.
